(19) United States
(12) Patent Application Publication
(10) Pub. No.: US 2023/0390178 A1
(43) Pub. Date: Dec. 7, 2023

(54) Skin Care Compositions and Methods of Use

(71) Applicant: Colgate-Palmolive Company, New York, NY (US)

(72) Inventors: Aaron COHEN, New Brunswick, NJ (US); Junhong MAO, Plainsboro, NJ (US); Thomas BOYD, Metuchen, NJ (US)

(73) Assignee: Colgate-Palmolive Company, New York, NY (US)

(21) Appl. No.: 18/328,049

(22) Filed: Jun. 2, 2023

Related U.S. Application Data
(60) Provisional application No. 63/348,630, filed on Jun. 3, 2022.

(51) Int. Cl. A61K 8/67; A61K 8/44; A61K 8/73; A61Q 19/00

(52) U.S. Cl. CPC A61K 8/676; A61K 8/675; A61K 8/44; A61K 8/735; A61Q 19/00

(57) ABSTRACT

The disclosure relates to skin care compositions comprising an effective amount of one or more hydrophilic vitamins and an effective amount of a low-molecular weight hyaluronic acid or an alkali metal hyaluronate (<avg. 75 kDa) (e.g., avg. 35-avg. 60 kDA) (e.g., about avg. 50 kDA), as well as to methods of using and of making these compositions.

### CROSS-REFERENCE TO RELATED PATENT APPLICATIONS

This application claims priority to U.S. Appl. No. 63/348,630, titled SKIN CARE COMPOSITIONS AND METHODS OF USE and filed Jun. 3, 2022, the content of which is incorporated herein by reference in its entirety for all purposes.

### FIELD

The disclosure relates to skin care compositions comprising one or more hydrophilic vitamins and a low-molecular weight hyaluronic acid or an alkali metal hyaluronate (<avg. 75 kDa) (e.g., avg. 35-avg. 60 kDA) (e.g., about avg. 50 kDA), as well as to methods of using and of making these compositions.

### BACKGROUND

The efficient topical delivery of hydrophilic ingredients, including hydrophilic vitamins such as ascorbic acid (vitamin C), into the viable regions of the skin has long been a challenging task. For example, pure ascorbic acid is a very hydrophilic active and this can limit its transport through the lipid channels that separate the keratinocytes of the outermost stratum corneum, and which forms the main barrier to active delivery. While some hydrophilic vitamins can be “derivatized” in order to increase their hydrophobicity, and thereby become a more permeable alternative, these derivatives lack the proven efficacy of topically applied hydrophilic vitamins, such as ascorbic acid.

While skin care compositions are commonly used to deliver active ingredients, the effect may be transient or ineffective as active agents may not be able to permeate various layers of the skin. This challenge may be even more pronounced with various hydrophilic ingredients used as the topical active ingredients in a number of skin care compositions. Therefore, there is a need to enhance the topical delivery of hydrophilic ingredients, such as ascorbic acid, enabling efficient delivery to the viable regions of the skin past the protective barrier layers of the skin.

### BRIEF SUMMARY

It has been surprisingly found that there is a specific molecular weight dependence of hyaluronic acid (“HA”) that can enhance and increase the depth of skin permeation of hydrophilic vitamins (e.g., ascorbic acid). At defined lower molecular weights (<average of 75 kDa) (e.g., average of 35 kDa-average of 60 kDA) (e.g., about average of 50 kDA) hyaluronic acid can have the effect of unexpectedly increasing the uptake and permeation of topical hydrophilic vitamins in the skin.

Hyaluronic acid (also called hyaluronan or hyaluronate), is an anionic, non-sulfated glycosaminoglycan (GAG) widely distributed throughout connective tissues of vertebrates, being the most abundant glycosaminoglycan of higher molecular weight in the extracellular matrix of soft periodontal tissues.

In one aspect the disclosure provides a skin care composition (Composition 1.0) comprising:
a. an effective amount of one or more hydrophilic vitamin(s) (e.g., ascorbic acid or ascorbyl glucoside) (e.g., vitamin B);
b. an effective amount of hyaluronic acid (HA) or an alkali metal hyaluronate with an average molecular weight (MW) of less than or equal to 75 kDa (e.g., from 35 kDa-60 kDA), (e.g., about 50 kDA).

For example, the disclosure contemplates any of the following compositions (unless otherwise indicated, values are given as percentage of the overall weight of the composition):
1.1 The skin care composition of Composition 1.0, wherein the hyaluronic acid or alkali metal hyaluronate has an average molecular weight of about 30 to about 70 kDa or about 30 to about 60 kDa. For instance, the hyaluronic acid and/or alkali metal hyaluronate may be adapted to have an average molecular weight of about 30 to about kDa, about 30 to about 55 kDa, about 30 to about 50 kDa, about 30 to about 45 kDa, about 30 to about 40 kDa, about 30 to about 35 kDa; from about 35 to about 75 kDa, about 35 to about 60 kDa, about 35 to about 55 kDa, about 35 to about 50 kDa, about 35 to about 45 kDa, about 35 to about 40 kDa, about 35 to about 35 kDa; from about 40 to about 70 kDa, about 40 to about 60 kDa, about 40 to about 55 kDa, about 40 to about 50 kDa, about 40 to about 45 kDa; from about 45 to about 70 kDa, about 45 to about 60 kDa, about 45 to about 55 kDa, about 45 to about 50 kDa; from about 50 to about 70 kDa, about 50 to about 60 kDa, about 50 to about 55 kDa, or any range or subrange thereof.
1.2 The preceding composition, wherein the hyaluronic acid or alkali metal hyaluronate polymer has an average molecular weight of 50 kDa (e.g., about 50 kDa).
1.3 Any of the preceding compositions, wherein the composition comprises the hyaluronic acid or alkali metal hyaluronate (e.g., sodium hyaluronate) in an amount of 0.01 to 10 wt. %, e.g., 0.01 to 5 wt. %, 0.05 to 5 wt. %, 0.1 to 2 wt. %, or 0.1 to 1.5 wt. %, or about 1 wt. %, or about 0.5 wt. %, or about 0.25 wt. %, based on the weight of the oral care composition. The amount of hyaluronic acid and/or alkali metal hyaluronate in the skin care composition may be from about 0.5 to about 5 wt. %, based on the total weight of the skin care composition. For example, the total amount of hyaluronic acid and/or alkali metal hyaluronate in the skin care composition may be from about 0.5 to about 5 wt. %, about 0.5 to about 4 wt. %, about 0.5 to about 3 wt. %, about 0.5 to about 2 wt. %, about 0.5 to about 1.5 wt. %, about 0.5 to about 1 wt. %; from about 0.75 to about 5 wt. %, about 0.75 to about 4 wt. %, about 0.75 to about 3 wt. %, about 0.75 to about 2 wt. %, about 0.75 to about 1.5 wt. %; from about 1 to about 5 wt. %, about 1 to about 4 wt. %, about 1 to about 3 wt. %, about 1 to about 2 wt. %, about 1 to about 1.5 wt. %; from about 1.5 to about 5 wt. %, about 1.5 to about 4 wt. %, about 1.5 to about 3 wt. %, about 1.5 to about 2 wt. %; from about 2 to about 5 wt. %, about 2 to about 4 wt. %, about 2 to about 3 wt. %; from about 3 to about 5 wt. %, about 3 to about 4 wt. %, or any range or subrange thereof, based on the total weight of the skin care composition.
1.4 The preceding composition, wherein the composition comprises the hyaluronic acid or alkali metal hyaluronate (e.g., sodium hyaluronate) in an amount of 0.05 wt. % to 5 wt. % based on the weight of the oral care composition.
1.5 The preceding composition, wherein the composition comprises the hyaluronic acid or alkali metal hyaluronate (e.g., sodium hyaluronate) in an amount of 0.025 wt. % to 2 wt. % based on the weight of the oral care composition.
1.6 Any of the preceding compositions, wherein the composition comprises the hyaluronic acid or alkali metal hyaluronate (e.g., sodium hyaluronate) in an amount of about 1% by wt. based on the weight of the oral care composition.
1.7 Any of the preceding skin care compositions, wherein the hydrophilic vitamin comprises vitamin C.
1.8 The skin care composition of 1.7, wherein the vitamin C is in the form selected from the group consisting of: ascorbic acid, L-ascorbic acid, ascorbyl glucoside, calcium ascorbate, calcium 1-ascorbate dihydrate, magnesium ascorbate, potassium ascorbate, magnesium L-ascorbyl phosphate (also referred to as: magnesium ascorbate phosphate or ascorbic acid phosphate magnesium salt), L-ascorbic acid 2-phosphate sesquimagnesium salt hydrate, (+) sodium L-ascorbate, dehydro-1-(+)-ascorbic acid dimer, sodium ascorbyl phosphate (also referred to as: ascorbic acid phosphate sodium salt, sodium 1-ascorbyl phosphate, 2-phospho-L-ascorbic acid trisodium salt, L-ascorbic acid 2-phosphate trisodium salt or sodium L-ascorbyl-2-phosphate), ascorbic acid-2-glucoside, ascorbyl dipalmitate, ascorbyl methylsilanol pectinate, ascorbyl stearate, disodium ascorbyl sulfate, ascorbyl 6-palmitate, calcium ascorbyl phosphate, ascorbyl acetate, ascorbyl propionate, ascorbyl stearate, ascorbyl palmitate, ascorbyl dipalmitate, ascorbic acid polypeptide, ethyl ascorbyl ether, ascorbyl ethyl silanol pectinate, and combinations thereof.
1.9 The skin care composition of 1.8, wherein the vitamin C is ascorbic acid (e.g., L-ascorbic acid).
1.10 The skin care composition of 1.8, wherein the vitamin C is ascorbyl glucoside.
1.11 The skin care composition of any of 1.8-1.10, wherein the vitamin C (e.g., ascorbic acid or ascorbyl glucoside) is present in an amount from 1% to 25%, e.g., from 5% to 25%, or from 10% to 25%, or from 15% to 25%, or about 20%, by weight of the composition.
1.12 Any of the preceding skin care compositions wherein the hydrophilic active ingredient comprises a B-complex vitamin (e.g., thiamin, riboflavin, niacin, vitamin B₃, vitamin B₆, folic acid, vitamin B₁₂, pantothenic acid, and biotin).
1.13 The preceding skin care composition, wherein the B-complex vitamin is selected from the group consisting of: thiamin, riboflavin, niacin, vitamin B₃, vitamin B₆, folic acid, vitamin B₁₂, pantothenic acid, biotin, and combinations thereof.
1.14 Any of the preceding skin care compositions, wherein the hydrophilic ingredient comprises vitamin B₃ represented by the following formula:

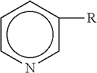

wherein R is —CONH2 (i.e., niacinamide), —COOH (i.e., nicotinic acid) or —CH2OH (i.e., nicotinyl alcohol); a derivative thereof, and/or a salt thereof.
1.15 The preceding skin care composition, wherein the vitamin B3 is selected from the group consisting of: nicotinic acid esters, non-vasodilating esters of nicotinic acid, nicotinyl amino acids, nicotinyl alcohol esters of carboxylic acids (e.g., straight-chain or branched chain, cyclic or acyclic, saturated or unsaturated (including aromatic), and substituted or unsubstituted, nicotinic acid N-oxide and niacinamide N-oxide, tocopherol nicotinate and inositol hexanicotinate. Examples of derivatives of niacinamide include nicotinyl amino acids, derived, for example, from the reaction of an activated nicotinic acid compound (e.g., nicotinic acid azide or nicotinyl chloride) with an amino acid, and nicotinyl alcohol esters of organic carboxylic acids (e.g., Cl-C18). Specific examples of such derivatives include nicotinuric acid and nicotinyl hydroxamic acid, which have the following chemical structures:

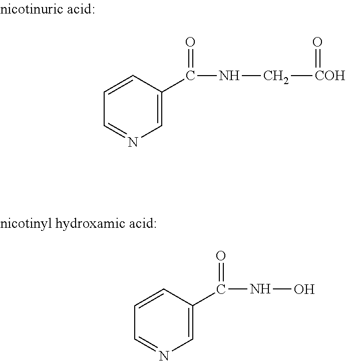

Additional derivatives of niacinamide includes nicotinyl alcohol esters, nicotinyl alcohol esters of the carboxylic acids salicylic acid, acetic acid, glycolic acid, palmitic acid and the like. Further examples of derivatives of niacinamide include 2-chloronicotinamide, 6-aminonicotinamide, 6-methylnicotinamide, n-methyl-nicotinamide, n,n-diethylnicotinamide, n-(hydroxymethyl)-nicotinamide, quinolinic acid imide, nicotinanilide, n-benzylnicotinamide, n-ethylnicotinamide, nifenazone, nicotinaldehyde, isonicotinic acid, methyl isonicotinic acid, thionicotinamide, nialamide, 1-(3-pyridylmethyl) urea, 2-mercaptonicotinic acid, nicomol, niaprazine, and combinations thereof.
1.16 Any of the preceding compositions, wherein the vitamin B3 compound is present in an amount from 1 to 25 wt. %, e.g., from 5 to 25 wt. %, or from 10 to 25 wt. %, or from 15 to 25 wt. %, or from 1 to 10 wt. %, or from 1 to 5 wt. %, or about 20 wt. %, or about 10 wt. %, or about 5 wt. %, or about 4 wt. %, or any range or subrange formed therefrom, all by weight of the composition. The total amount of vitamin B present in the skin care composition may be from about 0.1 to about 10 wt. %, based on the total weight of the skin care composition. For instance, the skin care composition may comprise a total amount of vitamin B from about 0.1 to about 5 wt. %, about 0.1 to about 4 wt. %, about 0.1 to about 3 wt. %, about 0.1 to about 2 wt. %, about 0.1 to about 1 wt. %, about 0.1 to about 0.7 wt. %; from about 0.3 to about 5 wt. %, about 0.3 to about 4 wt. %, about 0.3 to about 3 wt. %, about 0.1 to about 2 wt. %, about 0.3 to about 1 wt. %, about 0.3 to about 0.7 wt. %; from about 0.5 to about 5 wt. %, about 0.5 to about 4 wt. %, about 0.5 to about 3 wt. %, about 0.5 to about 2 wt. %, about 0.5 to about 1 wt. %, about 0.5 to about 0.7 wt. %; from about 0.7 to about 5 wt. %, about 0.7 to about 4 wt. %, about 0.7 to about 3 wt. %, about 0.7 to about 2 wt. %, about 0.7 to about 1 wt. %; from about 1 to about 10 wt. %, about 1 to about 8 wt. %, about 1 to about 6 wt. %, about 1 to about 5 wt. %, about 1 to about 4 wt. %, about 1 to about 3 wt. %, about 1 to about 2 wt. %, about 1 to about 1.5 wt. %, about 1 to about 1.25 wt. %; from about 1.25 to about 10 wt. %, about 1.25 to about 8 wt. %, about 1.25 to about 6 wt. %, about 1.25 to about 5 wt. %, about 1.25 to about 4 wt. %, about 1.25 to about 3 wt. %, about 1.25 to about 2 wt. %; from about 1.5 to about 10 wt. %, about 1.5 to about 8 wt. %, about 1.5 to about 6 wt. %, about 1.5 to about 5 wt. %, about 1.5 to about 4 wt. %, about 1.5 to about 3 wt. %, about 1.5 to about 2 wt. %; from about 2 to about 10 wt. %, about 2 to about 8 wt. %, about 2 to about 6 wt. %, about 2 to about 5 wt. %, about 2 to about 4 wt. %, about 2 to about 3 wt. %; from about 3 to about 10 wt. %, about 3 to about 8 wt. %, about 3 to about 6 wt. %, about 3 to about 5 wt. %, about 3 to about 4 wt. %; from about 4 to about 10 wt. %, about 4 to about 8 wt. %, about 4 to about 6 wt. %, about 4 to about 5 wt. %; from about 5 to about 10 wt. %, about 5 to about 8 wt. %, about 5 to about 6 wt. %, or any range or subrange thereof, based on the total weight of the skin care composition.
1.17 The skin care composition of any of the preceding compositions, wherein the vitamin B3 compound comprises niacinamide.
1.18 Any of the preceding skin care compositions, wherein the composition comprises water.
1.19 The preceding skin care compositions, wherein the amount of water is from 25 to 90 wt. %, e.g., about 30 wt. %, about 40 wt. %, about 50 wt. %, about 60 wt. %, about 70 wt. %, about 80 wt. %, about 90 wt. %, or any range or subrange thereof, based on the total weight of the skin care composition. For example, the total amount of water present in the skin care composition may be from about 25 to about 96 wt. %, about 40 to about 96 wt. %, about 50 to about 96 wt. %, about 60 to about 96 wt. %, about 70 25 to about 96 wt. %, about 80 to about 96 wt. %, about 85 to about 96 wt. %; from about 25 to about 90 wt. %, about 40 to about 90 wt. %, about 50 to about 90 wt. %, about 60 to about 90 wt. %, about 70 to about 90 wt. %, about 80 to about 90 wt. %, about 85 to about 90 wt. %; from about 25 to about 85 wt. %, about 40 to about 85 wt. %, about 50 to about 85 wt. %, about 60 to about 85 wt. %, about 70 to about 85 wt. %; from about 25 to about 80 wt. %, about 40 to about 80 wt. %, about 50 to about 80 wt. %, about 60 to about 80 wt. %, about 70 to about 80 wt. %; from about 25 to about 70 wt. %, about 40 to about 70 wt. %, about 50 to about 70 wt. %, about 60 to about 70 wt. %; from about 25 to about 60 wt. %, about 40 to about 60 wt. %, about 50 to about 60 wt. %; from about 25 to about 50 wt. %, about 40 to about 50 wt. %, about 50 to about 50 wt. %, about 60 to about 50 wt. %, or any range or subrange thereof, based on the total weight of the skin care composition.
1.20 Any of the preceding skin care compositions, wherein the skin care composition further comprises a gelling agent, an additional antioxidant, a fragrance, or a combination thereof.
1.21 Any of the preceding skin care compositions, wherein the skin care composition is topically delivered to the skin in the form selected from: a serum, a cream, a moisturizer, a mask, a cleanser, oil, salve, lotion, gel, ointment, paste a facial make-up, a toner, an essence, and a balm (e.g., lip balm).
1.22 Any of the preceding skin care compositions, wherein the skin care composition further comprises an additional vitamin selected from: vitamin D, vitamin E, vitamin K, and combinations thereof.
1.23 Any of the preceding skin care compositions, wherein the composition comprises one or more amino acids selected from: arginine, lysine, serine, citrulline, ornithine, creatine, histidine (e.g., a thiourea derivative of histidine, such as ergothioneine), diaminobutanoic acid, diaminoproprionic acid, alanine, aminobutyrate, asparagine, cysteine, cystine, glutamine, glycine, hydroxyproline, isoleucine, leucine, methionine, phenylalanine, proline, serine, taurine, threonine, tryptophan, tyrosine, valine and salts thereof, and combinations thereof. The skin care composition may include one or more of amino acid(s) in an amount from about 0.01 to about 5 wt. %, based on the total weight of the skin care composition. In some embodiments, the skin care composition may include an amino acid in amount from about 0.01 to about 2 wt. %, about 0.01 to about 1.7 wt. %, about 0.01 to about 1.4 wt. %, about 0.01 to about 1.2 wt. %, about 0.01 to about 1 wt. %, about 0.01 to about 0.8 wt. %, about 0.01 to about 0.6 wt. %, about 0.01 to about 0.4 wt. %, about 0.01 to about 0.2 wt. %, about 0.01 to about 0.1 wt. %, about 0.01 to about 0.05 wt. %; from about 0.05 to about 3 wt. %, about 0.05 to about 2 wt. %, about 0.05 to about 1.7 wt. %, about 0.05 to about 1.4 wt. %, about 0.05 to about 1.2 wt. %, about 0.05 to about 1 wt. %, about 0.05 to about 0.8 wt. %, about 0.05 to about 0.6 wt. %, about 0.05 to about 0.4 wt. %, about 0.05 to about 0.2 wt. %, about 0.05 to about 0.1 wt. %; from about 0.1 to about 3 wt. %, about 0.1 to about 2 wt. %, about 0.1 to about 1.7 wt. %, about 0.1 to about 1.4 wt. %, about 0.1 to about 1.2 wt. %, about 0.1 to about 1 wt. %, about 0.1 to about 0.8 wt. %, about 0.1 to about 0.6 wt. %, about 0.1 to about 0.4 wt. %, about 0.1 to about 0.2 wt. %; from about 0.3 to about 3 wt. %, about 0.3 to about 2 wt. %, about 0.3 to about 1.7 wt. %, about 0.3 to about 1.4 wt. %, about 0.3 to about 1.2 wt. %, about 0.3 to about 1 wt. %, about 0.3 to about 0.8 wt. %, about 0.3 to about 0.6 wt. %; from about 0.6 to about 3 wt. %, about 0.6 to about 2 wt. %, about 0.6 to about 1.7 wt. %, about 0.6 to about 1.4 wt. %, about 0.6 to about 1.2 wt. %, about 0.6 to about 1 wt. %; from about 1 to about 3 wt. %, about 1 to about 2 wt. %, about 1 to about 1.7 wt. %, about 1 to about 1.4 wt. %, about 1 to about 1.2 wt. %; from about 1.5 to about 3 wt. %, about 1.5 to about 2 wt. %, about 2 to about 3 wt. %, or any range or subrange thereof, based on the total weight of the skin care composition.
1.24 The total amount of amino acid(s) present in the skin care composition may be from about 0.1 to about 7 wt. %, based on the total weight of the skin care composition. For example, the total amount of amino acid(s) in the skin care composition may be from about 0.1 to about 6 wt. %, about 0.1 to about 5 wt. %, about 0.1 to about 3 wt. %, about 0.1 to about 2 wt. %, about 0.1 to about 1.7 wt. %, about 0.1 to about 1.4 wt. %, about 0.1 to about 1.2 wt. %, about 0.1 to about 1 wt. %, about 0.1 to about 0.8 wt. %, about 0.1 to about 0.6 wt. %, about 0.1 to about 0.4 wt. %, about 0.1 to about 0.2 wt. %; from about 0.3 to about 7 wt. %, about 0.3 to about 5 wt. %, about 0.3 to about 3 wt. %, about 0.3 to about 2 wt. %, about 0.3 to about 1.7 wt. %, about 0.3 to about 1.4 wt. %, about 0.3 to about 1.2 wt. %, about 0.3 to about 1 wt. %, about 0.3 to about 0.8 wt. %, about 0.3 to about 0.6 wt. %; from about 0.6 to about 7 wt. %, about 0.6 to about 5 wt. %, about 0.6 to about 3 wt. %, about 0.6 to about 2 wt. %, about 0.6 to about 1.7 wt. %, about 0.6 to about 1.4 wt. %, about 0.6 to about 1.2 wt. %, about 0.6 to about 1 wt. %; from about 1 to about 7 wt. %, about 1 to about 5 wt. %, about 1 to about 3 wt. %, about 1 to about 2 wt. %, about 1 to about 1.7 wt. %, about 1 to about 1.4 wt. %, about 1 to about 1.2 wt. %; from about 1.5 to about 7 wt. %, about 1.5 to about 5 wt. %, about 1.5 to about 3 wt. %, about 1.5 to about 2 wt. %, from about 2 to about 7 wt. %, about 2 to about 5 wt. %, about 2 to about 3 wt. %; from about 3 to about 7 wt. %, about 3 to 5 wt. %, about 4 to about 7 wt. % about 4 to 6 wt. %, or any range or subrange thereof, based on the total weight of the skin care composition.
1.25 The preceding skin care composition, wherein the composition comprises taurine, arginine and glycine.
1.26 The preceding skin care composition, wherein the taurine:arginine:glycine are in a weight ratio of (1.5-69):(0.5-40):(0.5-1.5), respectively. For instance, the skin care composition may be formulated to have a weight ratio of taurine to arginine to glycine (taurine:arginine:glycine) from about 85:45:1 to about 45:24:1. In some embodiments, the skin care composition has a weight ratio of taurine to arginine to glycine from about 80:42:1 to about 50:30:1; from about 75:40:1 to about 55:29:1; from about 70:37:1 to about 60:31:1, or any range or subrange thereof.
1.27 The preceding composition, wherein the taurine:arginine:glycine are in a weight ratio of about (65):(34):(1), respectively.
1.28 Any of the preceding skin care compositions, wherein the composition comprises an additional ingredient selected from carnosine, cica (asiaticoside), magnesium sulfate, saccharide isomerate, a keto-carotenoid (e.g., astaxanthin), nicotinamide adenine dinucleotide (NAD), and a combination of two or more thereof.
1.29 Any of the preceding skin care compositions further comprising a topically acceptable carrier.
1.30 Any of the preceding skin care compositions wherein one or more ingredients are encapsulated in a delivery vesicle (e.g., a multilamellar vesicle (MLV)).
1.31 The preceding skin care composition, wherein the MLV comprises liposome structuring amounts of polyglyceryl-10 dioleate and at least one of polyglyceryl-10 dilinoleate or polyglyceryl-10 dipalmitate in a weight ratio of polyglyceryl-10 dioleate to the at least one of polyglyceryl-10 dilinoleate or polyglyceryl-10 dipalmitate of from 300:1 to 5:1
1.32 Any of the preceding skin care compositions, wherein the MLV comprises one or more ingredients selected from: polyglyceryl-10 dilinoleate, polyglyceryl-10 dipalmitate, polyglyceryl-10 dioleate, Caprylic/Capric Triglyceride, and a combination of two or more thereof.
1.33 Any of the preceding skin care compositions, wherein the skin care composition is delivered using a transdermal injection (e.g., mesotherapy).
1.34 Any of the preceding skin care compositions comprising:
   an effective amount of hyaluronic acid (e.g., from 30 kDa-60 kDa) (e.g., about kDa); and
   an effective amount of ascorbic acid (e.g., L-ascorbic acid).
1.35 Any of the preceding skin care compositions comprising:
   from 1 to 5 wt. % of hyaluronic acid at about 50 kDa; and
   from 1 to 25 wt. % of ascorbic acid (e.g., L-ascorbic acid), wherein all weight percentages are based on the total weight of the skin care composition.
1.36 Any of the preceding skin care compositions comprising:
   an effective amount of hyaluronic acid (e.g., from 30 kDa-60 kDa) (e.g., about kDa); and
   an effective amount of ascorbyl glucoside.
1.37 Any of the preceding skin care compositions comprising:
   from 1 to 5 wt. % of hyaluronic acid at about 50 kDa; and
   from 1 to 25 wt. % of ascorbyl glucoside, wherein all weight percentages are based on the total weight of the skin care composition.
1.38 Any of the preceding skin care compositions comprising:
   an effective amount of hyaluronic acid (e.g., from 30 kDa-60 kDa) (e.g., about kDa); and
   an effective amount of ascorbic acid (e.g., L-ascorbic acid) and ascorbyl glucoside.
1.39 Any of the preceding skin care compositions, wherein the skin care composition is delivered using a transdermal injection (e.g., mesotherapy).
1.40 Any of the preceding skin care compositions wherein one or more ingredients are encapsulated in a delivery vesicle (e.g., a multilamellar vesicle (MLV)).
1.41 Any of the preceding skin care compositions, wherein the skin care composition is in the form selected from: a serum, a cream, a moisturizer, a mask, a cleanser, oil, salve, lotion, gel, ointment, paste a facial make-up, and a balm (e.g., lip balm)
1.42 Any of the preceding compositions, wherein the composition comprises a thickener.
1.43 Any of the preceding skin care compositions, wherein the thickener comprises a gum, optionally selected from xanthan gum, carrageenan, and a combination of two or more thereof.
1.44 Any of the preceding skin care compositions, wherein the composition comprises a humectant, optionally wherein the humectant is selected from glycerin, sorbitol, and a combination of two or more thereof.
1.45 Any of the preceding skin care compositions, comprising an additional antioxidant(s) selected from the group consisting of: flavonoids, phytoalexins, carnosine, tocotrienol, a lipoic acid, a carotenoid, melatonin, and any combinations thereof.
1.46 Any of the preceding skin care compositions, further comprising an anti-inflammatory agent selected from the group consisting of: dexamethasone, prednisolone, corticosterone, budesonide, sulfasalazine, mesalamine, cetirizine, diphenylhydramine, antipyrine, methyl salicylate, loratadine, thymol, carvacrol, bisabolol, allantoin, eucalyptol, phenazone, propyphenazone, a nonsteroidal anti-inflammatory drug NSAID, and any combinations thereof.
1.47 Any of the preceding compositions further comprising an anti-itching agent, wherein the agent is selected from the group consisting of methyl sulphonyl methane, sodium bicarbonate, calamine, allantoin, kaolin, peppermint, tea tree oil, camphor, menthol, hydrocortisone, and any combination of two or more thereof.
1.48 Any of the preceding skin care compositions, wherein the skin care composition comprises:
   alpha-tocopherol;
   pyrrolidone carboxylic acid (“PCA”) and/or a salt thereof (e.g. sodium PCA);
   niacinamide;
   hyaluronic acid (e.g., average molecular weight of 50 kDa); and
   ascorbic acid and/or ascorbyl glucoside.
1.49 Any of the preceding skin care compositions, wherein the skin care composition comprises:
   alpha-tocopherol;
   pyrrolidone carboxylic acid (“PCA”) and/or a salt thereof (e.g. sodium PCA);
   hyaluronic acid (e.g., average of 50 kDa); and
   vitamin B.
1.50 Any of the preceding skin care compositions, wherein the pyrrolidone carboxylic acid and/or a salt thereof comprises L-pyrrolidone carboxylic acid and/or a salt thereof. In some embodiments, the skin care composition comprises pyrrolidone carboxylic acid and/or salt(s) thereof in an amount from about 0.1 to about 5 wt. %, based on the total weight of the personal care composition. For example, the total amount of the pyrrolidone carboxylic acid and/or salt(s) thereof present in the skin care composition may be from about 0.1 to about 5 wt. %, about 0.1 to about 4 wt. %, about 0.1 to about 3 wt. %, about 0.1 to about 2 wt. %, about 0.1 to about 1 wt. %, about 0.1 to about 0.7 wt. %; from about 0.3 to about 5 wt. %, about 0.3 to about 4 wt. %, about 0.3 to about 3 wt. %, about 0.1 to about 2 wt. %, about 0.3 to about 1 wt. %, about 0.3 to about 0.7 wt. %; from about 0.5 to about 5 wt. %, about 0.5 to about 4 wt. %, about 0.5 to about 3 wt. %, about 0.5 to about 2 wt. %, about 0.5 to about 1 wt. %, about 0.5 to about 0.7 wt. %; from about 0.7 to about 5 wt. %, about 0.7 to about 4 wt. %, about 0.7 to about 3 wt. %, about 0.7 to about 2 wt. %, about 0.7 to about 1 wt. %; from about 1 to about 5 wt. %, about 1 to about 4 wt. %, about 1 to about 3 wt. %, about 1 to about 2 wt. %, about 1 to about 1.5 wt. %, about 1 to about 1.25 wt. %; from about 1.25 to about 5 wt. %, about 1.25 to about 4 wt. %, about 1.25 to about 3 wt. %, about 1.25 to about 2 wt. %; from about 1.5 to about 5 wt. %, about 1.5 to about 4 wt. %, about 1.5 to about 3 wt. %, about 1.5 to about 2 wt. %; from about 2 to about 5 wt. %, about 2 to about 4 wt. %, about 2 to about 3 wt. %; from about 3 to about 5 wt. %, about 3 to about 4 wt. %, or any range or subrange thereof, based on the total weight of the skin care composition.
1.51 Any of the preceding skin care compositions, wherein the hyaluronic acid is acetylated.
1.52 Any of the preceding skin care compositions, wherein the composition comprises sodium hyaluronate.
1.53 Any of the preceding skin care compositions, wherein the hydrophilic vitamin is a polar compound comprising one or more ionic groups.
1.54 Any of the preceding skin care compositions, wherein the hydrophilic vitamin has a degree of wettability where the surface of compound has a contact angle (θ)<90°.
1.55 Any of the preceding skin care compositions, wherein the amount of hyaluronic acid (e.g., average of 30 kDa-60 kDa) (e.g., average of 50 kDa) is effective to increase or enhance the uptake and permeation of the hydrophilic vitamins in the skin.
1.56 Any of the preceding skin care compositions, wherein the skin care composition is free or substantially free of hydroquinone. In some cases, the skin care composition comprises about 4 wt. % or less, about 3 wt. % or less, about 2 wt. % or less, about 1 wt. % or less, about 0.5 wt. % or less, about 0.1 wt. % or less, based on the total weight of the skin care composition, of hydroquinone.
1.57 Any of the preceding skin care compositions, wherein the skin care composition is free or substantially free of ethanol. In some cases, the skin care composition comprises about 4 wt. % or less, about 3 wt. % or less, about 2 wt. % or less, about 1 wt. % or less, about 0.5 wt. % or less, about 0.1 wt. % or less, based on the total weight of the skin care composition, of ethanol.
1.58 Any of the preceding compositions, wherein the amount of hyaluronic acid (e.g., average of 30 kDa-60 kDa) (e.g., average of 50 kDa) is effective to deliver the hydrophilic vitamin further than the stratum corneum layer of the skin (e.g., effective to deliver the hydrophilic vitamin to the dermis or epidermal layers).
1.59 Any of the preceding compositions, wherein the hydrophilic vitamin has a Log P of less than 0 (e.g., ≤−1) (e.g., between −0.1 to −1). Log P refers to the constant defined by log 10 (Partition Coefficient). The Partition Coefficient (P) may be determined by the formula: P=[organic]/[aqueous]. The partition coefficient (P) describes the propensity of a neutral (uncharged) compound to dissolve in an aqueous portion versus an organic portion of a biphasic system.
1.60 Any of the preceding skin care compositions, wherein the skin care is in a single phase.
1.61 Any of skin care compositions 1.0-1.57, wherein the skin care composition is biphasic.
1.62 Any of the preceding skin care compositions wherein the skin care composition is biphasic and comprises a first phase and second phase, wherein the first phase comprises hyaluronic acid and wherein the second phase comprises the hydrophilic vitamin (e.g., ascorbic acid).
1.63 The skin care composition of 1.60, wherein the first phase comprising hyaluronic acid is applied to the skin prior to the application of the second phase comprising the hydrophilic vitamin (e.g., ascorbic acid).

The present disclosure provides, in another aspect, a skin care composition (Composition 2.0), e.g., skin care composition for use in inhibiting, treating or reducing damage to the skin caused reactive oxygen species. In one aspect, the skin care composition for use can be any of Composition 1.0 et seq.

The present disclosure provides, in another aspect, a method of inhibiting, treating or reducing damage to the skin caused reactive oxygen (Method 1.0), wherein the method comprises administering to a subject in need thereof an effective amount of any of Composition 1.0 et seq.
1.1 Method 1.0 where the skin care composition (e.g., any of Composition 1.0 et seq) is delivered via a transdermal injection.
1.2 Method 1.0, wherein the skin care composition is topically delivered to the skin using a serum, a cream, a moisturizer, a mask, a cleanser, oil, salve, lotion, gel, ointment, paste, a facial make-up, a toner, an essence, or a balm (e.g., lip balm).

In another aspect, the present disclosure provides a method of depositing a topically active compound on the skin (Method 2.0), comprising applying an effective amount of any of skin care compositions disclosed herein, e.g., any of Compositions 1 et seq., to the skin.
2.1 Method 2.0 where the skin care composition (e.g., any of Composition 1.0 et seq) is delivered via a transdermal injection.
2.2 Method 2.0, wherein the skin care composition is topically delivered to the skin using a serum, a cream, a moisturizer, a mask, a cleanser, oil, salve, lotion, gel, ointment, paste, a facial make-up, a toner, an essence, or a balm (e.g., lip balm).

In another aspect, the present disclosure provides a method selected from the following (Method 3.0):
vitalization of skin cells in a subject in need thereof;
enhancing cellular energy in skin cells (e.g., dermal cells) in a subject in need thereof;
reduction or inhibition of cellular damage (e.g., from reactive oxygen species, UV light damage) in a subject in need thereof;
cellular detoxification (e.g., in dermal cells) in a subject in need thereof; and a combination of two or more thereof.
wherein any of the above methods comprises applying an effective amount of any of skin care compositions disclosed herein, e.g., any of Compositions 1 et seq., to the skin.
3.1 Method 3.0 where the skin care composition (e.g., any of Composition 1.0 et seq) is delivered via a transdermal injection.
3.2 Method 3.0, wherein the skin care composition is topically delivered to the skin using a serum, a cream, a moisturizer, a mask, a cleanser, oil, salve, lotion, gel, ointment, paste a facial make-up, a toner, an essence, or a balm (e.g., lip balm).

In another aspect, the present disclosure provides a method selected from the following (Method 4.0):
treatment of redness and/or reduction skin pore size in a subject in need thereof;
treatment of fine lines, wrinkles, or scars in a subject in need thereof;
treatment to increase smoothness, hydration, and/or elasticity of the skin in a subject in need thereof;
reduction or inhibition of cellular damage (e.g., from reactive oxygen species,
UV light damage) in a subject in need thereof;
cellular detoxification (e.g., in dermal cells) in a subject in need thereof; and a combination of two or more thereof.
wherein any of the above methods comprises applying an effective amount of any of skin care compositions disclosed herein, e.g., any of Compositions 1 et seq., to the skin.
   4.1 Method 4.0 where the skin care composition (e.g., any of Composition 1.0 et seq) is delivered via a transdermal injection.
   4.2 Method 4.0, wherein the skin care composition is topically delivered to the skin using a serum, a cream, a moisturizer, a mask, a cleanser, oil, salve, lotion, gel, ointment, paste a facial make-up, a toner, an essence, or a balm (e.g., lip balm).

In some embodiments, the skin care composition, e.g., any of Composition 1.0 et seq., comprises an oil selected from sunflower seed oil, olive oil, shear butter, jojoba oil, almond oil, grape seed oil, rose hip seed oil, mink oil, castor oil, soybean oil, mineral oil, and a combination thereof, optionally wherein the oil is sunflower seed oil.

In some embodiments, the skin care composition, e.g., any of Composition 1.0 et seq., comprises a thickener. In some embodiments, the thickener comprises a gum, optionally selected from xanthan gum, carrageenan and a combination thereof.

In some embodiments, the skin care composition, e.g., any of Composition 1.0 et seq., comprises a humectant, optionally wherein the humectant is selected from glycerin, sorbitol, and a combination thereof.

In some embodiments, the skin care composition, e.g., any of Composition 1.0 et seq., comprises water.

In some embodiments, the skin care composition, e.g., any of Composition 1.0 et seq., is free or substantially free of sulfate. In some cases, the skin care composition comprises about 4 wt. % or less, about 3 wt. % or less, about 2 wt. % or less, about 1 wt. % or less, about 0.5 wt. % or less, about 0.1 wt. % or less, based on the total weight of the skin care composition, of sulfate and/or sulfate based compound.

In some embodiments, the skin care composition, e.g., any of Composition 1.0 et seq., further comprises, a gelling agent, an additional antioxidant, a fragrance, or a combination thereof.

In some embodiments, the composition, e.g., any of Composition 1.0 et seq., is in the form selected from: a serum, a cream, a moisturizer, a mask, a cleanser, a facial make-up, and a balm (e.g., lip balm).

The composition of the present disclosure, e.g., any of Composition 1.0 et seq., may be any type of skin care composition. In certain embodiments, the composition is any composition that can be formulated into topical skin care formulations suitable for application to skin. Examples of such compositions include, but are not limited to: serums, creams, moisturizers, masks, cleanser (e.g., facial cleansers), a facial make-up, balms (e.g., lip balm), and cosmetics. The composition can comprise a single phase or can be a multi-phase system, for example a system comprising a polar phase and an oil phase, optionally in the form of a stable emulsion. In certain aspects, skin care compositions of the disclosure are formulated in a manner suitable for topically injection.

The compositions of the disclosure (e.g., any of Composition 1.0 et seq) can be liquid, semi-solid or solid. The formulation can be provided in any suitable container such as an aerosol can, tube or container with a porous cap, roll-on container, bottle, container with an open end, etc.

In some aspects, water may be present in the compositions of the disclosure, e.g., any of Composition 1.0 et seq. Water employed in the preparation of commercial skin care compositions should be deionized and free of organic impurities. When it is incorporated into the formulation, water can make up the balance of the compositions and includes about 10 to about 90 wt. %, or about 10 to about 80 wt. %, by weight of the skin care compositions. This amount of water includes the free water which is added plus that amount which is introduced with other materials such as glycerin, sorbitol, or any components of the disclosure.

In some embodiments, the skin care compositions of the disclosure, e.g., any of Composition 1.0 et seq., can include one or more pH adjusters. When incorporated in the formulation, the pH adjuster can include, but is not limited to, lactic acid, sodium hydroxide, and/or citric acid.

In some embodiments, the skin care compositions, e.g., any of Composition 1.0 et seq., may further comprise a betaine zwitterionic surfactant. In some aspects, the betaine zwitterionic surfactant may be a C₈-C₁₆ aminopropyl betaine, e.g., cocamidopropyl betaine.

In some embodiments, the skin care compositions described herein, e.g., any of Composition 1.0 et seq., may further comprises a non-ionic block copolymer is selected from Poloxamer 338, Poloxamer 407, Poloxamer, 237, Poloxamer, 217, Poloxamer 124, Poloxamer 184, Poloxamer 185, and a combination of two or more thereof.

As used herein, the term “effective amount” means the quantity of an active ingredient and/or skin care composition required to increase or enhance the uptake and permeation of one or more topical hydrophilic vitamins in the skin.

As used herein, “substantially free” of a material may refer to a composition where the material is present in an amount of less than 0.1 wt. %, less than 0.05 wt. %, less than 0.01 wt. %, less than 0.005 wt. %, less than 0.001 wt. %, or less than 0.0001 wt. %, based on a total weight of the skin care composition.

As used herein, “skin care composition” is meant to refer to a composition for which the intended use can include promotion or improvement of health, cleanliness, odor, appearance, or attractiveness of skin. For example, skin care compositions can include serum, a cream, a moisturizer, a mask, a cleanser, oil, salve, lotion, gel, ointment, paste, a facial make-up, a toner, an essence, and a balm (e.g., lip balm). Skin care compositions of the disclosure, e.g., any of Composition 1.0 et seq., can be applied topically to the skin by a user or consumer. In some aspects, skin care compositions of the disclosure can be applied by injection (e.g., subcutaneous or dermal injection). In certain aspects, skin care composition is intended to refer to a product for use at home or in a professional setting.

In certain aspects, the skin care compositions of the disclosure (e.g., any of Composition 1.0 et seq) can comprises one or more topically active compounds selected from: antibacterial agents, vitamins, medicaments, fragrance materials, antioxidants, other skin-care ingredients, and combinations of two or more thereof.

As used herein, unless otherwise specified, “Vitamin E” refers to a family of four isomers of tocopherols and four isomers of tocotrienols. All eight isomers of vitamin E have a 6-chromanol ring structure and a side chain. The four tocopherols include fully saturated side chains and include alpha-tocopherol, gamma-tocopherol, beta-tocopherol, and delta-tocopherol. The four tocotrienols include unsaturated side chains and include alpha-tocotrienol, gamma-tocotrienol, beta-tocotrienol, and delta-tocotrienol. As used herein, unless otherwise specified, the term “vitamin E” may refer to any one or more of the eight isomers. For example, as used herein, vitamin E may be or include one or more of alpha-tocopherol, gamma-tocopherol, beta-tocopherol, delta-tocopherol, alpha-tocotrienol, gamma-tocotrienol, beta-tocotrienol, delta-tocotrienol, or any combination thereof. In at least one implementation, the vitamin E includes at least one of the four tocopherols. It should be appreciated that the vitamin E and/or the isomers thereof may be or include natural forms of vitamin E, synthetic forms of vitamin E, or combinations thereof. Any one or more of the isomers of vitamin E may be in the “d” form, the “l” form, or combinations thereof. In some embodiments, vitamin E is vitamin E acetate or Vitamin E succinate. In some embodiments, vitamin E is vitamin E acetate.

As used herein, “Vitamin C” may be ascorbic acid or derivatives thereof. Ascorbic acid exists as two enantiomers commonly denoted “1” (for “levo”) and “d” (for “dextro”). The “1” isomer is the one most often encountered. Ascorbic acid is also referred to as L(+)-ascorbic acid or 1-ascorbic acid. The ascorbic acid derivatives may be or include, but are not limited to, L-ascorbic acid, calcium ascorbate, calcium 1-ascorbate dihydrate, magnesium ascorbate, potassium ascorbate, magnesium L-ascorbyl phosphate (also referred to as: magnesium ascorbate phosphate or ascorbic acid phosphate magnesium salt), L-ascorbic acid 2-phosphate sesquimagnesium salt hydrate, (+) sodium L-ascorbate, dehydro-1-(+)-ascorbic acid dimer, sodium ascorbyl phosphate (also referred to as: ascorbic acid phosphate sodium salt, sodium 1-ascorbyl phosphate, 2-phospho-L-ascorbic acid trisodium salt, L-ascorbic acid 2-phosphate trisodium salt or sodium L-ascorbyl-2-phosphate), ascorbic acid-2-glucoside, ascorbyl dipalmitate, ascorbyl methylsilanol pectinate, ascorbyl stearate, disodium ascorbyl sulfate, ascorbyl 6-palmitate, calcium ascorbyl phosphate, ascorbyl acetate, ascorbyl propionate, ascorbyl stearate, ascorbyl palmitate, ascorbyl dipalmitate, ascorbyl glucoside, ascorbic acid polypeptide, ethyl ascorbyl ether, ascorbyl ethyl silanol pectinate, or the like, or combinations thereof.

For example, the skin care composition includes a Vitamin C selected from ascorbyl glucoside, ascorbic acid-2-glucoside, ascorbyl acetate, and a combination of two or more thereof. In certain embodiments, the skin care composition comprises from ascorbyl glucoside, ascorbic acid-2-glucoside, or a combination thereof. In at least one embodiment, the one or more Vitamin C comprises ascorbyl glucoside.

The total amount of vitamin C, e.g., ascorbic acid or derivatives thereof, present in the skin care composition may be from about 0.1 to about 5 wt. %, based on the total weight of the skin care composition. For instance, the skin care composition may comprise a total amount of vitamin C from about 0.1 to about 5 wt. %, about 0.1 to about 4 wt. %, about 0.1 to about 3 wt. %, about 0.1 to about 2 wt. %, about 0.1 to about 1 wt. %, about 0.1 to about 0.7 wt. %; from about 0.3 to about 5 wt. %, about 0.3 to about 4 wt. %, about 0.3 to about 3 wt. %, about 0.1 to about 2 wt. %, about 0.3 to about 1 wt. %, about 0.3 to about 0.7 wt. %; from about 0.5 to about 5 wt. %, about 0.5 to about 4 wt. %, about 0.5 to about 3 wt. %, about 0.5 to about 2 wt. %, about 0.5 to about 1 wt. %, about 0.5 to about 0.7 wt. %; from about 0.7 to about 5 wt. %, about 0.7 to about 4 wt. %, about 0.7 to about 3 wt. %, about 0.7 to about 2 wt. %, about 0.7 to about 1 wt. %; from about 1 to about 5 wt. %, about 1 to about 4 wt. %, about 1 to about 3 wt. %, about 1 to about 2 wt. %, about 1 to about 1.5 wt. %, about 1 to about 1.25 wt. %; from about 1.25 to about 5 wt. %, about 1.25 to about 4 wt. %, about 1.25 to about 3 wt. %, about 1.25 to about 2 wt. %; from about 1.5 to about 5 wt. %, about 1.5 to about 4 wt. %, about 1.5 to about 3 wt. %, about 1.5 to about 2 wt. %; from about 2 to about 5 wt. %, about 2 to about 4 wt. %, about 2 to about 3 wt. %; from about 3 to about 5 wt. %, about 3 to about 4 wt. %, or any range or subrange thereof, based on the total weight of the skin care composition.

As used herein, “vitamin D” refers to a group of fat-soluble secosteroids responsible for increasing intestinal absorption of calcium, magnesium, and phosphate, and many other biological effects. The two major forms are vitamin D₂ or ergocalciferol, and vitamin D₃ or cholecalciferol. Vitamin D includes vitamin D₁ (mixture of molecular compounds of ergocalciferol with lumisterol), vitamin D₂ (ergocalciferol), vitamin D₃ (cholecalciferol), vitamin D₄ (22-dihydroergocalciferol), and vitamin D₅ (sitocalciferol).

As used herein, “vitamin K” is a group of compounds with a common chemical structure of 2-methyl-1,4-naphthoquinone. Vitamin K plays a role in blood clotting, bone metabolism, and regulating blood calcium levels. Vitamin K includes vitamin K₁ (phylloquinone) and vitamin K₂ (menaquinone). Vitamin K₂ have unsaturated isoprenyl side chains and are designated as MK-4 through MK-13, based on the length of their side chain.

Optional ingredients that may be included in the skin care composition of the invention include solvents; water-soluble alcohols such as C₂₋₈ alcohols including ethanol; glycols including propylene glycol, dipropylene glycol, tripropylene glycol and mixtures thereof; glycerides including mono-, di- and triglycerides; medium to long chain organic acids, alcohols and esters; surfactants including emulsifying and dispersing agents; amino acids including glycine; structurants including thickeners and gelling agents, for example polymers, silicates and silicon dioxide; emollients; fragrances; and colorants including dyes and pigments.

The skin care compositions of the disclosure, e.g., any of Composition 1.0 et seq., may optionally contain emollients in any desired amount to achieve a desired emollient effect. Emollients are known in the art and are used to impart a soothing effect on the skin. Non-volatile emollients are preferable. Classes of non-volatile emollients include non-silicone and silicone emollients, e.g., Poly silicone-11. Non-volatile, non-silicone emollients include C₁₂₋₁₅ alkyl benzoate. The non-volatile silicone material can be a polyethersiloxane, polyalkyarylsiloxane or polyethersiloxane copolymer. An illustrative non-volatile silicone material is phenyl trimethicone or polymethylsilsesquioxane. Examples include, but are not limited to, PPG-14 butyl ether, PPG-3 myristyl ether, secondary alcohol ethoxylates, stearyl alcohol, stearic acid and salts thereof, glyceryl monoricinoleate, isobutyl palmitate, glyceryl monostearate, isocetyl stearate, sulphated tallow, oleyl alcohol, propylene glycol, isopropyl laurate, mink oil, sorbitan stearate, cetyl alcohol, hydrogenated castor oil, stearyl stearate, hydrogenated soy glycerides, isopropyl isostearate, hexyl laurate, dimethyl bras sylate, decyl oleate, diisopropyl adipate, n-dibutyl sebacate, diisopropyl sebacate, 2-ethyl hexyl palmitate, isononyl isononanoate, isodecyl isononanoate, isotridecyl isononanoate, 2-ethyl hexyl palmitate, 2-ethyl hexyl stearate, Di-(2-ethyl hexyl)adipate), Di-(2-ethyl hexyl)succinate, isopropyl myristate, isopropyl palmitate, isopropyl stearate, octacosanol, butyl stearate, glyceryl monostearate, polyethylene glycols, oleic acid, triethylene glycol, lanolin, castor oil, sunflower seed oil, acetylated lanolin alcohols, acetylated lanolin, petrolatum, isopropyl ester of lanolin, fatty acids, mineral oils, butyl myristate, isostearic acid, palmitic acid, PEG-23 oleyl ether, olelyl oleate, isopropyl linoleate, cetyl lactate, lauryl lactate, myristyl lactate, quaternised hydroxy alkyl, aminogluconate, vegetable oils, isodecyl oleate, isostearyl neopentanoate, myristyl myristate, oleyl ethoxy myristate, diglycol stearate, ethylene glycol monostearate, myristyl stearate, isopropyl lanolate, paraffin waxes, glycyrrhizic acid, hydrocyethyl stearate amide. In some embodiments, the composition comprises an oil selected from sunflower seed oil, olive oil, shear butter, jojoba oil, almond oil, grape seed oil, rose hip seed oil, mink oil, castor oil, soybean oil, mineral oil, and a combination thereof. In certain embodiment, the composition comprises sunflower seed oil.

The skin care compositions of the disclosure, e.g., any of Composition 1.0 et seq., may optionally include one or more humectants. Humectants can reduce evaporation and also contribute towards preservation by lowering water activity and can also impart desirable sweetness or flavor to compositions. Illustrative humectants may be or include, but are not limited to, glycerin, propylene glycol, polyethylene glycol, sorbitol, xylitol, or the like, or any mixture or combination thereof. In some embodiment, the humectant is selected from glycerin, sorbitol and a combination thereof. In certain embodiment, the humectant is glycerin.

The skin care compositions of the disclosure, e.g., any of Composition 1.0 et seq., may optionally include thickeners. Illustrative thickeners may be or include, but are not limited to, colloidal silica, fumed silica, a cross-linked polyvinylpyrrolidone (PVP) polymer, cross-linked polyvinylpyrrolidone (PVP), or the like, or mixtures or combinations thereof. In some embodiments, the thickening system includes a cross-linked polyvinylpyrrolidone (PVP) polymer. Illustrative thickeners may also be or include, but are not limited to, carbomers (e.g., carboxyvinyl polymers), carrageenans (e.g., Irish moss, carrageenan, iota-carrageenan, etc.), high molecular weight polyethylene glycols, cellulosic polymers, hydroxyethylcellulose, carboxymethylcellulose, and salts thereof (e.g., CMC sodium), natural gums (e.g., karaya, xanthan, gum arabic, and tragacanth), colloidal magnesium aluminum silicate, or the like, or mixtures or combinations thereof. In some embodiments, the thickener comprises or is a gum, optionally selected from xanthan gum, carrageenan, and a combination thereof.

The skin care compositions of the disclosure, e.g., any of Composition 1.0 et seq., may optionally include one or more gelling agents. Examples of gelling agents include, but are not limited to, waxes, esters of fatty acid and fatty alcohol, triglycerides, partially or fully hydrogenated soybean oil, partially or fully hydrogenated castor oil, other partial or fully hydrogenated plant oils, stearyl alcohol, or other cosmetically acceptable materials, which are solid or semi-solid at room temperature and provide a consistency suitable for application to the skin.

The skin care compositions of the disclosure, e.g., any of Composition 1.0 et seq., may optionally have one or more antioxidants. For example, one or more antioxidants may be added to the composition to act as ingredient protectants and for maintenance of long-term stability of the composition. Examples of optional antioxidants include, but are not limited to citric acid, butylated hydroxytoluene, pentaerythrityl tetra-di-t-butyl hydroxyhydrocinnamate.

The skin care compositions of the disclosure, e.g., any of Composition 1.0 et seq., may optionally polymeric materials for thickening, such as polyamides, cellulose derivatives (e.g., hydroxypropylcellulose, hydroxypropyl methyl cellulose, etc.) and natural or synthetic gums, such as polyglycerides including agar, agarose, pectin, or guars or mixtures or combinations thereof. One class of materials worthy of attention for thickening a water-immiscible phase comprises derivatives of hydrolysed starch or other polysaccharides, including in particular esterified dextrins, such as dextrin palmitate. A further class of polymers that is particularly directed to structuring an oil phase containing a silicone oil comprises polysiloxane elastomers. Suspending agents such as silicas or clays such as bentonite, montmorillonite or hectorite, including those available under the trademark Bentone can also be employed to thicken liquid compositions according to the invention. The composition can be thickened with non-polymeric organic gellants, including selected dibenzylidene alditols (e.g., dibenzylidene sorbitol).

The skin care compositions of the disclosure, e.g., any of Composition 1.0 et seq., may optionally include a fragrance. Any fragrance suitable for skin care use may be incorporated into the skin care composition of the disclosure. Fragrances tend to be relatively volatile aroma compounds which are capable of entering the gas phase at skin surface temperature.

The skin care compositions of the disclosure, e.g., any of Composition 1.0 et seq., may be manufactured using methods known in the art. Typically, the ingredients are combined and optionally heated where components need to be melted. The components are mixed. Desirably, volatile materials such as fragrant materials are incorporated in the composition in the latter stages of a mixing cycle in order to avoid volatilization thereof. After mixing, the composition may be poured directly into the dispensers and the container capped to preserve the product until use.

### EXAMPLES

### Example 1—Topical Delivery of Ascorbic Acid in Combination with Hyaluronic Acid

To observe the uptake enhancement properties of various weights of hyaluronic acid, samples of porcine ear skin are first “pre-treated” with a 1% aqueous solution of the appropriate hyaluronic acid. Control samples are treated with water only. Following this pre-treatment, samples were then treated with a market-based skin care composition containing 20% by wt. of ascorbic acid and placed on a Franz cell (Logan Instruments, DHC-6T) for a 24-hour diffusion period. Following treatment, excess product was removed from the skin surface and the stratum corneum was removed from the skin via tape stripping twenty times. The remaining skin consists of the viable epidermis and the dermis layers, which were then homogenized and quantitatively analyzed for ascorbic acid content via an Agilent 1200 Series with UV-vis detector high performance liquid chromatography (HPLC). For each experiment, an “enhancement factor” was calculated for the viable skin regions. The “enhancement factor” is calculated as follows:

Enhancement Factor=100*(((penetrated ascorbic acid in samples pre-treated with HA)/(penetrated ascorbic acid in control samples only treated with water))−1)

This calculation allows for ease of comparison between the many different experiments that are conducted, the results of which are as follows:

TABLE 1

| Sample and | |
|---|---|
| Molecular Wt. of | Enhancement |
| HA Used (kDa) | Factor |
| Sample A | 19.4 |
| (HA: average of 3.6 kDa) | |
| Sample B | 35.9 |
| (HA: average of 20 kDa) | |
| Sample C | 49.2 |
| (HA: average of 50 kDa) | |
| Sample D | 21.7 |
| (HA: average of 360 kDa) | |
| Sample E | −7.0 |
| (HA: average of 1,000-1,400 kDa) | |
| Sample F | −7.5 |
| (Acetylated HA: average of 20 kDa) | |

Table 1 shows the enhancement factor for the various samples, each sample incorporating a different molecular weight HA or chemically modified HA.

Without being bound by theory, one of skill in the art may initially expect there to be a linear relationship where the samples receiving a treatment with a lower molecular weight HA would exhibit a greater enhancement of topical ascorbic acid delivery. And this expectation may be due to the potential for increased dermal absorption of lower molecular weight HA. However, as demonstrated by Table 1, the relationship is not linear. Surprisingly, the enhancement of topical ascorbic acid delivery was the highest using Sample C (˜50 kDa hyaluronic acid). Unexpectedly, both Sample A and Sample B both demonstrate decreased enhancement of ascorbic acid despite incorporating hyaluronic at average molecular weights of 3.6 kDa and 20 kDa, respectively, that were considerably lower than Sample C. Without being bound by theory, while lower molecular weight variants are believed to absorb more easily into the skin, it is possible that these lower molecular weight variants are also more prone to degradation which may limit their effectiveness. Alternatively, lower molecular weight hyaluronic acid may be less effective at absorbing water. Therefore, in order for the hyaluronic acid to enhance or effectively deliver ascorbic acid, the molecular weight must be low enough to readily absorb into the skin, but not so low that it is also prone to degradation and limited in its ability to absorb water.

Notably, the two samples treated with higher molecular weight HA, Samples D and E, both demonstrated a decreased ability to enhance the topical delivery of ascorbic acid. With respect to Sample E, the enhancement effect is actually negative. This should be interpreted to mean that samples pre-treated with the hyaluronic acid with an average molecular weight of 1,000-1,400 kDa have a reduced level of topical ascorbic acid delivery compared to the control samples that are pre-treated with only water. Visually, skin samples pre-treated with hyaluronic acid having an average molecular weight of 1,000-1,400 kDa appear to develop a film on their surface. Without being bound by theory, this observation could be because the molecular weight of the HA used in Sample E is so large that it is not absorbed into the skin and may simply remain on the skin surface to form a film. Again, without being bound by theory, this film forming effect may explain why Sample E demonstrates a negative value with respect to the topical delivery of ascorbic acid.

To further explore the possible mode of action of how HA enhances the topical delivery of ascorbic acid, Sample F is treated with average molecular weight 20 kDa acetylated hyaluronic acid. Although its low molecular weight of the acetylated hyaluronic acid suggests that it should readily absorb into the skin, without being bound by theory, it is possible that the partial capping of the polar hydroxyl groups with acetyl groups may significantly decrease the overall water binding abilities. As demonstrated in Table 1, similar to much larger molecular weight hyaluronic acid (e.g., Sample E), acetylated hyaluronic acid (having an average molecular weight of 20 kDa) also hinders the topical delivery of pure ascorbic acid even though the molecular weight of the acetylated hyaluronic acid is about 50 times lower than that used in Sample E. Again, without being bound by theory, this may indicate that the strong water binding ability of (non-acetylated) HA is critical in its ability to enhance the topical delivery of ascorbic acid. Moreover, it is clear from the range of molecular weight hyaluronic acid in Table 1 that the relationship with the delivery of ascorbic acid, and the molecular weight of the hyaluronic acid, is not linear. Again, without being bound by theory, it is possible that this unexpected relationship is due to hyaluronic acid with an average molecular weight of 50 kDa has a molecular weight that is low enough to be readily absorbed into the skin, and where the molecular weight is not so low that its water binding properties are greatly diminished.

The composition of the “market-based skin care composition” contained the following list of ingredients: Cyclopentasiloxane, Ascorbic Acid, Ethylhexyl Hydroxystearate, Polysilicone-11, Tocopherol, Isosorbide Dicaprylate, Polymethylsilsesquioxane, Hexylresorcinol, Silybum Marianum Fruit Extract, Hordeum Distichon (Barley) Extract, Citrus Aurantium Dulcis (Orange) Oil, Silica, Phellodendron Amurense Bark Extract, Santalum Album (Sandalwood) Wood Extract.

### Example 2—Topical Delivery of Ascorbyl Glucoside in Combination with Hyaluronic Acid

Ascorbyl glucoside is a derivative of ascorbic acid. However, it is believed that ascorbyl glucoside also has several properties that one of skill in the art may believe could impede its topical delivery into the skin. For example, compared to ascorbic acid, ascorbyl glucoside is both larger (MW of 338 g/mol for ascorbyl glucoside vs 176 g/mol for ascorbic acid) and more polar (Log P of −2.5 for ascorbyl glucoside vs Log P of −1.85 for ascorbic acid) and, thus, more hydrophilic.

The topical delivery of ascorbyl glucoside was carried out using the same experimental procedure as described in Example 1. Briefly, 20 wt. % of ascorbyl glucoside was loaded into the base formula of the “market-based skin care composition” described in Example 1, such that the ascorbyl glucoside containing skin care composition contained a total of 20 wt. % of ascorbyl glucoside relative to the total weight of the ascorbyl glucoside containing skin care composition. Note, however, that the active ingredients were removed from the market-based skin care composition, such that the ascorbyl glucoside containing skin care composition did not include ascorbic acid, tocopherol, hexylresorcinol or Silybum Marianum Fruit Extract.

Porcine ear skin explants were treated with either pure water (control) or a 1 wt. % aqueous solution of a hyaluronic acid (“HA”) with an average molecular weight of 50 kDa, wherein the solution contained 1 wt. % aqueous solution of a hyaluronic acid with the remainder being water. Subsequently, the skin pieces were treated with the ascorbyl glucoside containing skin care composition and then placed on a Franz diffusion cell for roughly 24 hours. Following this period, the top twenty layers of the skin were removed using adhesive tape strips. These tape strip layers, plus the remainder of the skin, were all analyzed for ascorbyl glucoside content using high performance liquid chromatography (HPLC).

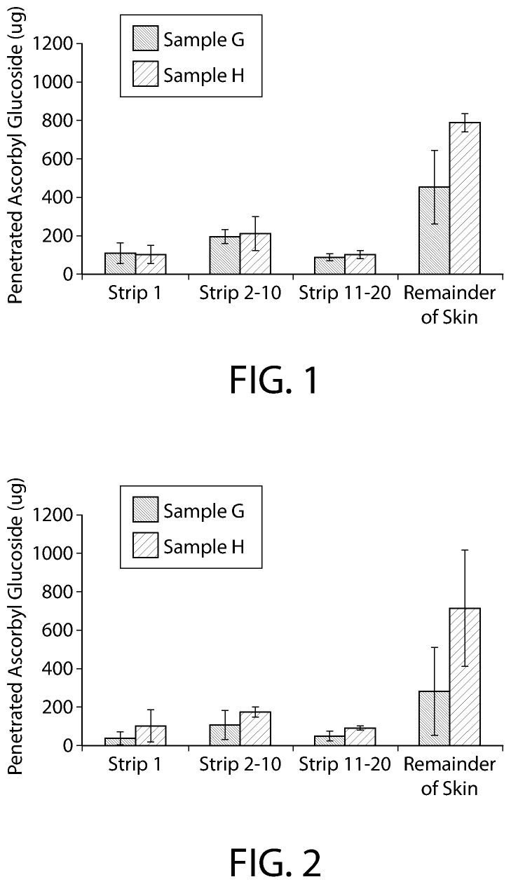

The results of each trial with Sample G and Sample H can be seen in Table 2 and FIGS. 1 and 2. In both cases, the trends are very similar. The region demonstrating the highest amount of ascorbyl glucoside is the “skin remainder,” also referred to as the “remainder of skin”. The “skin remainder” refers to the region of the skin beyond the stratum corneum, which is believed to approximately represent the viable regions of the skin as well as a proxy for enhanced topical delivery of a hydrophilic vitamin, such as ascorbyl glucoside. In both trials, there was a significantly higher level of ascorbyl glucoside in the skin samples subject to treatment with the 1 wt. % of hyaluronic acid solution (˜50 kDa) relative to the skin samples subject to treatment with only water (p<0.10). The absolute levels of ascorbyl glucoside in these regions is detailed in Table 2 with the levels of the units in the “Remainder of Skin” represented in micrograms.

TABLE 2

| | Sample G | Sample H |
|---|---|---|
| | (Skin samples | (Skin samples |
| | treated with | treated with 1% |
| | water and 20% | HA solution |
| | by wt. of | (~50 kDa) and |
| | ascorbyl | 20% by wt. of |
| | glucoside) | ascorbyl glucoside) |
| Trial 1 | 452 +/− 190 ug* | 787 +/− 207 ug* |
| (Absolute Levels of | | |
| Ascorbyl Glucoside in | | |
| “Remainder of Skin”) | | |
| Trial 2 | 286 +/− 227 ug** | 718 +/− 300 ug** |
| (Absolute Levels of | | |
| Ascorbyl Glucoside in | | |
| “Remainder of Skin”) | | |

*p value: ~0.055
**p value: ~0.061

The general observable trends were very similar to that seen for 20 wt. % pure ascorbic acid in an anhydrous base, used in Example 1, in which the skin sample was pre-treated with a 1 wt. % solution of hyaluronic acid having an average molecular weight of 50 kDa.

In summary, the results from Table 2 demonstrate that a 1 wt. % solution of hyaluronic acid having an average molecular weight of 50 kDa can significantly enhance the topical delivery of ascorbyl glucoside from a composition containing 20 wt. % of ascorbyl glucoside.

### Example 3—Topical Delivery of Carnosine in Combination with Hyaluronic Acid

The topical delivery of carnosine was carried out using the same experimental procedure as described in Examples 1 and 2. Briefly, a carnosine composition was prepared by incorporating carnosine into the market-based skin care composition” described in Example 1 in an amount such that the carnosine composition contained 5 wt. % of carnosine relative to the total weight of the carnosine composition. The active ingredients were removed from the market-based skin care composition, such that the carnosine skin care composition did not include ascorbic acid, tocopherol, hexylresorcinol or Silybum Marianum Fruit Extract.

Porcine ear skin explants (Sample I), which was treated with an aqueous solution of 1 wt. % of hyaluronic acid having an average molecular weight of 50 kDa with the remainder being water and the carnosine composition, were prepared. A control (Sample J) was prepared by treating porcine ear skin explant with water and the carnosine composition. Specifically, the porcine ear skin explant received either aqueous solution of 1 wt. % of hyaluronic acid having an average molecular weight of 50 kDa with the remainder being water or the water before being treated with the carnosine composition.

After the carnosine composition was applied to the porcine ear skin explant, the porcine ear skin explant was placed on a Franz diffusion cell for roughly 24 hours. Following this period, the top twenty layers of the skin were removed using adhesive tape strips. These tape strip layers, plus the remainder of the skin, were all analyzed for carnosine content using high performance liquid chromatography (HPLC).

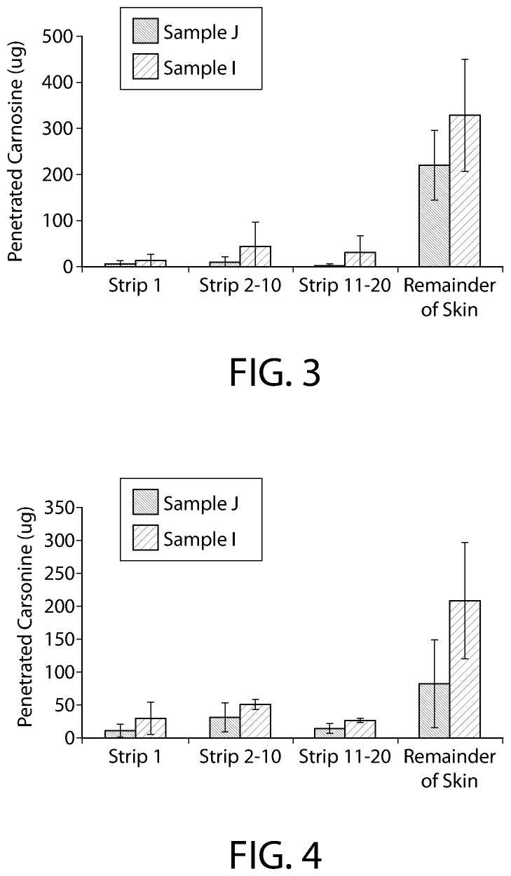

The results of the two trials, which evaluated the combination of the carnosine composition with the aqueous solution of a hyaluronic acid having an average molecular weight of 50 kDa as compared with the carnosine composition with water (control) can be seen in Table 3 (below) as well as in FIGS. 3 and 4.

TABLE 3

| | Sample I | |
|---|---|---|
| | (Skin Sample | Sample H |
| | with solution of | (Skin Sample |
| | 50 kDA HA and | water and |
| | the carnosine | the carnosine |
| | composition) | composition) |
| Trial 1 | 326.6 ± 123.1 μg | 220.5 ± 76.9 μg |
| (Absolute Level of | | |
| Carnosine in | | |
| “Remainder of Skin”) | | |
| Trial 2 | 206.7 ± 119.5 μg | 121.3 ± 41.3 μg |
| (Absolute Level of | | |
| Carnosine in | | |
| “Remainder of Skin”) | | |

The region demonstrating the highest amount of carnosine was the “skin remainder.” In both trials, there was a significantly higher level of carnosine in the skin samples subject to treatment with the 1 wt. % of hyaluronic acid solution (˜50 kDa) relative to the skin samples subject to treatment with only water.

The present disclosure has been described with reference to exemplary embodiments. Although a limited number of embodiments have been shown and described, it will be appreciated by those skilled in the art that changes may be made in these embodiments without departing from the principles and spirit of the preceding detailed description. It is intended that the present disclosure be construed as including all such modifications and alterations insofar as they come within the scope of the appended claims or the equivalents thereof.

### Example 4—A Non-Limiting, Example Skin Care Composition

A non-limiting example composition was prepared in accordance with aspects of the invention. The formulation for the exemplary composition is shown in Table 4 (below).

TABLE 4

| | Example |
|---|---|
| | Composition |
| US INCI Compound Name | (wt. %) |
| Ergothioneine | 0.3-0.9 |
| Amino Acids (Taurine, Arginine, and | 1.7-2.5 |
| Glycine) | |
| Alpha-tocopherol | 0.5-1.7 |
| Vitamin B3/Niacinamide | 1.7-2.5 |
| Carnosine | 0.5-1.7 |
| Sodium PCA (“Nalidone” 50% solution) | 0.5-1.7 |
| Hyaluronic Acid (low MW-50 kDa) | 0.5-1.7 |
| Nicotinamide Adenine dinucleotide | 0.001-0.05 |
| (NAD) and Magnesium sulfate | |

1-23. (canceled)

24. A skin care composition comprising:
one or more hydrophilic vitamin; and
a hyaluronic acid and/or a salt thereof having an average molecular weight of about 30 to about 70 kDa.

25. The skin care composition according to claim 24, wherein the average molecular weight of the hyaluronic acid and/or the salt thereof is from about 30 to about 60 kDa, optionally about 35 to about 60 kDa, optionally about 35 to about 55 kDa, optionally about 40 to about 60 kDa, optionally about 40 to about 55 kDa, or optionally about 45 to about 55 kDa.

26. The skin care composition according to claim 24, wherein the one or more hydrophilic vitamin comprises vitamin C, carnosine, vitamin B, or a combination of two or more thereof.

27. The skin care composition according to claim 26, wherein the vitamin C is selected from ascorbic acid, L-ascorbic acid, ascorbyl glucoside, calcium ascorbate, calcium 1-ascorbate dihydrate, magnesium ascorbate, potassium ascorbate, magnesium L-ascorbyl phosphate, L-ascorbic acid 2-phosphate sesquimagnesium salt hydrate, (+) sodium L-ascorbate, dehydro-1-(+)-ascorbic acid dimer, sodium ascorbyl phosphate, ascorbic acid-2-glucoside, ascorbyl dipalmitate, ascorbyl methylsilanol pectinate, ascorbyl stearate, disodium ascorbyl sulfate, ascorbyl 6-palmitate, calcium ascorbyl phosphate, ascorbyl acetate, ascorbyl propionate, ascorbyl stearate, ascorbyl palmitate, ascorbyl dipalmitate, ascorbic acid polypeptide, ethyl ascorbyl ether, ascorbyl ethyl silanol pectinate, and a combination of two or more thereof.

28. The skin care composition according to claim 26, wherein the vitamin C is selected from ascorbyl glucoside, ascorbic acid-2-glucoside, ascorbyl acetate, and a combination of two or more thereof.

29. The skin care composition according to claim 26, wherein the vitamin C comprises ascorbyl glucoside, ascorbic acid-2-glucoside, or a combination of two or more thereof.

30. The skin care composition according to claim 26, wherein the vitamin B comprises niacinamide.

31. The skin care composition according to claim 24, further comprising:
an amino acid component selected from arginine, lysine, serine, citrulline, ornithine, creatine, histidine (e.g., a thiourea derivative of histidine, such as ergothioneine), diaminobutanoic acid, diaminoproprionic acid, alanine, aminobutyrate, asparagine, cysteine, cystine, glutamine, glycine, hydroxyproline, isoleucine, leucine, methionine, phenylalanine, proline, serine, taurine, threonine, tryptophan, tyrosine, valine, and a combination of two or more thereof.

32. The skin care composition according to claim 30, wherein the amino acid component is selected from creatine, histidine, glutamine, glycine, taurine, and a combination thereof.

33. The skin care composition according to claim 30, wherein the amino acid component comprises taurine, arginine, and glycine.

34. The skin care composition according to claim 33, wherein the skin care composition has a weight ratio of the taurine to the arginine to the glycine (taurine:arginine:glycine) from about 85:45:1 to about 45:24:1.

35. The skin care composition according to claim 24, wherein the hyaluronic acid and/or the salt is selected from hyaluronic acid (HA), an alkali metal hyaluronate, and a combination of two or more thereof.

36. The skin care composition according to claim 24, wherein the hyaluronic acid and/or the salt comprises sodium hyaluronate.

37. The skin care composition according to claim 24, wherein the hydrophilic vitamin has a degree of wettability where the surface of compound has a contact angle (θ)<90°.

38. The skin care composition according to claim 24, wherein the amount of the hyaluronic acid and/or the salt thereof is effective to increase or enhance the uptake and permeation of the hydrophilic vitamins in the skin.

39. The skin care composition according to claim 24, wherein the amount of the hyaluronic acid and/or the salt thereof is effective to deliver the hydrophilic vitamin further than the stratum corneum layer of the skin.

40. The skin care composition according to claim 24, wherein at least one of the hydrophilic vitamin has a Log P of less than 0.

41. The skin care composition according to claim 24, wherein the hyaluronic acid and/or the salt thereof is present in an amount from about 0.01 to about 10 wt. %, based on the total weight of the skin care composition.

42. The skin care composition according to claim 24, wherein the one or more hydrophilic vitamin is present in an amount about 1 to 25 wt. %, based on the total weight of the skin care composition.

43. The skin care composition according to claim 24, comprising:
from about 1 to about 5 wt. % of the hyaluronic acid and/or the salt thereof, the hyaluronic acid and/or the salt thereof having an average molecular weight of about 50 kDa; and
from about 1 to about 25 wt. % of ascorbic acid, ascorbyl glucoside, or a combination thereof,
wherein all weight percentages are based on the total weight of the skin care composition.
